(19) United States
(12) Patent Application Publication
(10) Pub. No.: US 2009/0143793 A1
(43) Pub. Date: Jun. 4, 2009

(54) CONNECTOR FOR SURGICAL ANASTOMOSES

(75) Inventors: Leok Poh Chua, Singapore (SG); Dhanjoo Noshir Ghista, Singapore (SG); Yong Seng Tan, Singapore (SG)

Correspondence Address: BLAKELY SOKOLOFF TAYLOR & ZAFMAN LLP, 1279 OAKMEAD PARKWAY, SUNNYVALE, CA, 94085-4040, US

(73) Assignee: Nanyang Technological University, Singapore (SG)

(21) Appl. No.: 11/947,684

(22) Filed: Nov. 29, 2007

(51) Int. Cl. A61F 2/06; A61B 17/11

(52) U.S. Cl. 606/155; 623/1.36

(57) ABSTRACT

A connector for surgical anastomoses is disclosed. The connector has a sleeve with an inlet and an outlet. The inlet is for fluid connection with a first blood vessel. The inlet is generally co-axial with the first blood vessel. The outlet is for fluid connection with a second blood vessel. The outlet is generally orthogonal to the second blood vessel.

### TECHNICAL FIELD

This invention relates to a connector for surgical anastomoses and refers particularly, though not exclusively, to such a connector for coronary artery anastomoses.

### BACKGROUND

In cardiac surgery, anastomoses for coronary artery bypass grafting (CABG) were traditionally done with handsewn sutures. During CABG, one end of a graft conduit is sutured to a blood supply such as the aorta, while another end of the conduit is sutured to a target vessel such as a coronary artery. The conduit is typically a saphenous or other vein graft. Hand sewing the saphenous vein graft to the coronary artery is an extremely difficult and time-consuming task due to the smaller diameter of the coronary vessel (typically from 1 to 4 mm) compared with the diameter of the saphenous vein graft which is typically from 5 to 7 mm. Any imprecision when placing sutures between the coronary artery and the graft may lead to occlusion at the anastomosis site, causing severe flow impairment. Suturing also inevitably introduces vascular wall damage, slowing down healing of the anastomosis.

In addition to suturing the graft to the aorta, the graft also has to be sutured to the occluded coronary artery, preferably at a point distal to its occluded segment (distal anastomosis). To perform the surgery, the surgeon thus needs relatively unobstructed access to the anastomosis site within the patient. In less invasive surgical approaches, some of the major coronary arteries are not readily reached by the surgeon. This makes suturing either difficult or impossible for some coronary artery sites.

An additional problem with CABG is formation of thrombi and atherosclerotic lesions at and around the grafted coronary artery, which can result in reoccurrence of myocardial infarction.

There is therefore a need for a simpler way of performing coronary bypass surgery that can minimize dependence of surgical outcome on a surgeon's personal suturing skill. It is also desirable to dispense with suturing altogether. It is further desirable to minimize formation of thrombi and atherosclerotic lesions.

### SUMMARY

According to a first aspect, there is provided a connector for surgical anastomoses. The connector comprises a sleeve having an inlet and an outlet, the inlet being for fluid connection with a first blood vessel, the inlet being generally co-axial with the first blood vessel, and the outlet being for fluid connection with a second blood vessel, the outlet being generally orthogonal to the second blood vessel.

The connector may comprise smooth and continuous change from the inlet to the outlet. The inlet may be circular. The outlet may be elliptical. The outlet may flare outwardly. The sleeve may have a smooth internal wall. The connector may further comprise a circumferential groove on an outer wall of the sleeve adjacent the inlet for assisting securing the first blood vessel thereto. The connector may further comprise an attachment adjacent the outlet for attaching the connector to the second blood vessel. The attachment may comprise a docking ring. The docking ring may be elliptical. The docking ring may include a mechanical attachment selected from: a plurality of securing clips, claws, spring clips, spring claws, retaining rings, circlips, and split rings.

The mechanical attachment may comprise securing claws resiliency biased towards an open clamping position for securing the docking ring to the second blood vessel. The securing claws may be made of a shape memory material. The shape memory material may be Nitinol. The docking ring may further comprise at least one positioning magnet. The docking ring may be secured to the sleeve by spikes on the docking ring locking with holes on the sleeve.

### BRIEF DESCRIPTION OF THE DRAWINGS

In order that the present invention may be fully understood and readily put into practical effect, there shall now be described by way of non-limitative example only preferred embodiments of the present invention, the description being with reference to the accompanying illustrative drawings.

In the drawings:

FIG. 1 is a schematic cross-section view of a vein attached to a coronary artery via a connector;

FIG. 2 is a schematic perspective view of a sleeve in the connector of FIG. 1;

FIG. 3 is a schematic perspective view of an attachment device to be used with the sleeve of FIG. 2; and

FIG. 4 is an alternative embodiment of the sleeve of FIG. 2.

### DETAILED DESCRIPTION OF THE PREFERRED EMBODIMENTS

In order to determine the influence of distal anastomotic geometry on formation of atherosclerotic lesion in the graft and coronary artery (due to deleterious blood flow velocity and shear stress distributions), computational fluid dynamics and in-vitro experiments of blood flow in the aorta, graft and the occluded coronary arteries were performed. These have been published extensively in international journals.

Formation of thrombi and atherosclerotic lesions at and around the grafted coronary artery, which can result in reoccurrence of myocardial infarction, was found to be dependent on the geometry of the distal anastomosis, which can cause complex flow velocity and shear stress distributions that are contributory to intimal hyperplasia due to formation of atherosclerotic lesion within the graft and the coronary artery.

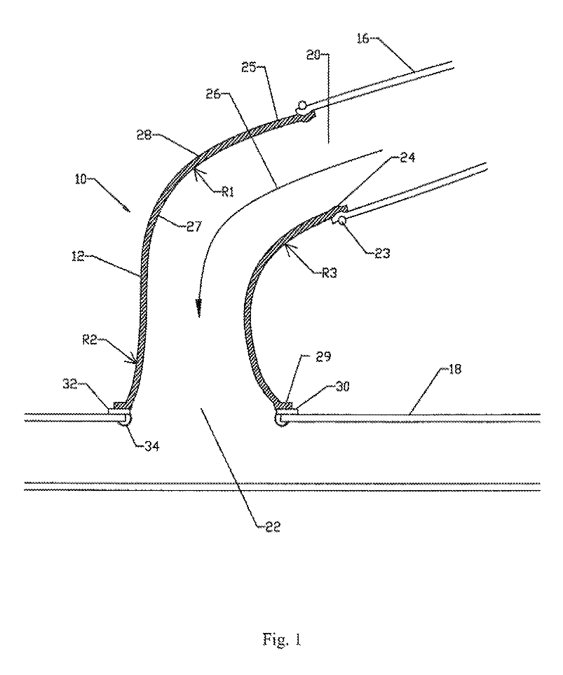

To optimise the geometry of the distal anastomosis, as shown in FIG. 1, there is provided a connector 10. In use, the connector 10 is in fluid connection with a first blood vessel 16 and a second blood vessel 18. In the case of CABG, the second blood vessel 18 would be a coronary artery while the first blood vessel 16 may be a saphenous vein graft. The connector 10 facilitates flow of oxygenated blood from the vein graft 16 into the coronary artery 18.

The connector 10 comprises a sleeve 12 having an inlet 20 and an outlet 22. The vein graft 16 is connected to the inlet 20, preferably by tying the vein graft 16 to the sleeve 12. A circumferential groove 24 on outer wall 25 of the sleeve 12 may be provided adjacent to the inlet 20 to secure the tying and prevent the vein graft 16 from slipping off the sleeve 12. The vein graft may be tied with a suture thread 23 against the groove 24. Compared to suturing, tying is a much quicker and simpler process and avoids the vascular wall damage that arises from suturing. Other mechanical methods and/or apparatus may be used such as, for example, circular clamps, spring clips, elastic bands, and so forth.

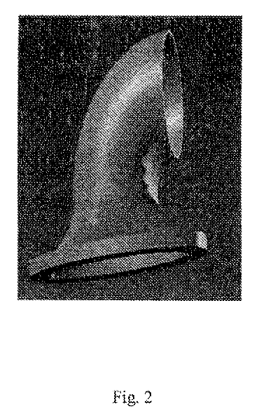

A perspective view of the sleeve 12 is given in FIG. 2. The sleeve 12 is preferably about 0.1 mm thick in general and made of a biocompatible plastics material such as a biocompatible polyurethane. Preferably, the outlet 22 is generally orthogonal to the coronary artery 18 while the inlet 20 is generally co-axial to the vein graft 16, so that blood flow is in the direction indicated by arrow 26 in FIG. 1. This is achieved by having a bend 28 in the sleeve 12. The bend 28 preferably has an outer radius of curvature R₁ ranging from 4.5 to 5.0 mm and an inner radius of curvature R₃ ranging from 1.2 to 1.6 mm to avoid thrombosis.

The inlet 20 may be circular in shape, with a diameter ranging from 3 to 4 mm, for securing the vein graft 16 thereto. The outlet 22 is preferably elliptical in shape, for accommodating a smooth transition from the greater diameter vein graft 16 to the smaller diameter coronary artery 18. The change from circular at inlet 20 to elliptical at outlet 22 is smooth and continuous with there being no disruption to smoothness of internal wall 27 of the sleeve 12 to avoid thrombosis. The elliptical outlet 22 may have a major axis of about 5 mm and a minor axis of about 1.4 mm. The outlet 22 may also have a rim 30 about 0.6 mm wide and about 0.4 mm thick.

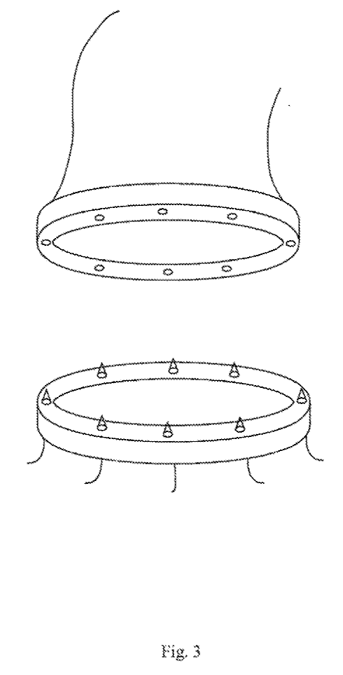

The connector 10 may further comprise an attachment device 30 to attach the sleeve 12 to the coronary artery 18. As shown in FIG. 3, the attachment device 30 comprises a docking ring 32 for engaging sleeve rim 29 at the elliptical outlet 22 of the sleeve 12. The docking ring 32 may be secured to the sleeve 12 by spikes 34 on the docking ring 32 that lock into holes 36 in the sleeve rim 29. Correct positioning may be assisted by magnetic force between the docking ring 32 and the sleeve 12, by making the docking ring 32 and the sleeve rim 29 from suitable magnetic materials and/or embedding micro-magnets in either or both of the docking ring 32 and the sleeve rim 29.

The docking ring 32 also has a mechanical attachment for securely attaching the docking ring 32 to the coronary artery 18. The mechanical attachment may be of any suitable nature of a form including, but not limited to, securing clips, securing claws, spring clips, spring claws, retaining rings, circlips, split rings and so forth. As shown, located on the docking ring 32 are a plurality of securing claws 34. The securing claws 34 are resiliently biased towards an open clamping position for securing the docking ring 32 to the coronary artery 18 as shown in FIG. 1. Each of the securing claws preferably has a diameter ranging from 40 to 70 μm. A minimum of two, but preferably three, securing claws are provided on each quadrant of the docking ring 32 to ensure secure attachment of the connector 10 to the coronary artery 18. Preferably, the securing claws 34 are made of a shape memory material such as a Nitinol alloy.

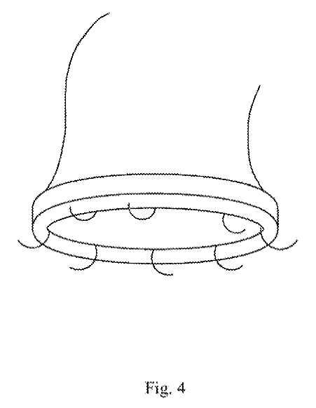

Alternatively, the securing claws 34 may be directly located on the sleeve rim 29, as shown in FIG. 4.

Prior to application, the securing claws 34 are preferably retracted or forced straight for easy insertion into the coronary artery 18. Upon insertion and activation of a release mechanism, the securing claws 34 spring back to their open clamping position, thereby securing the connector 10 to the coronary artery 18, as well as stretching open the incision to allow blood flow from the vein graft 16 through the connector 10 into the coronary artery 18. In this way, suturing is avoided and the disadvantages attendant with suturing are likewise eliminated. There is thus minimal interference to the blood flow with use of the connector 10, thereby reducing the risks of clotting at the anastomosis.

Providing the connector 10 between the vein graft 16 and the coronary artery 18 also improves hemodynamic performance of the anastomosis by improving the fluid flow pattern (and associated flow velocities and shear stresses) between the two blood vessels 16, 18. Also, kinks in the vein graft 16 that may obstruct blood flow in the vein graft 16 are minimized as a result of the sleeve 12 maintaining sufficient stand-off between the vein graft 16 and the coronary artery 18. This stand-off prevents the vein graft 16 from kinking and/or folding back upon itself, which may easily occur if it is directly sutured orthogonally to the coronary artery and the anastomosis site is compressed by surrounding tissue.

Whilst there has been described in the foregoing description preferred embodiments of the present invention, it will be understood by those skilled in the technology concerned that many variations or modifications in details of design or construction may be made without departing from the present invention.

What is claimed:

1. A connector for surgical anastomoses comprising:
a sleeve having an inlet and an outlet,
the inlet being for fluid connection with a first blood vessel, the inlet being generally co-axial with the first blood vessel, and
the outlet being for fluid connection with a second blood vessel, the outlet being generally orthogonal to the second blood vessel.

2. The connector of claim 1, comprising smooth and continuous change from the inlet to the outlet.

3. The connector of claim 2, wherein the inlet is circular.

4. The connector of claim 3, wherein the outlet is elliptical.

5. The connector of claim 4, wherein the outlet flares outwardly.

6. The connector of claim 1, wherein the sleeve has a smooth internal wall.

7. The connector of claim 1, further comprising a circumferential groove on an outer wall of the sleeve adjacent the inlet for assisting securing the first blood vessel thereto.

8. The connector of claim 1, further comprising an attachment adjacent the outlet for attaching the connector to the second blood vessel.

9. The connector of claim 8, wherein the attachment comprises a docking ring.

10. The connector of claim 3, wherein the docking ring is elliptical.

11. The connector of claim 9, wherein the docking ring includes a mechanical attachment selected from the group consisting of: a plurality of securing clips, claws, spring clips, spring claws, retaining rings, circlips, and split rings.

12. The connector of claim 11, wherein the mechanical attachment comprises securing claws resiliency biased towards an open clamping position for securing the docking ring to the second blood vessel.

13. The connector of claim 12, wherein the securing claws are made of a shape memory material.

14. The connector of claim 13, wherein the shape memory material is Nitinol.

15. The connector of claim 11, wherein the docking ring further comprises at least one positioning magnet.

16. The connector of claim 9, wherein the docking ring is secured to the sleeve by spikes on the docking ring locking with holes on the sleeve.
